(19) United States
(12) Patent Application Publication
(10) Pub. No.: US 2017/0065178 A1
(43) Pub. Date: Mar. 9, 2017

(54) MEASUREMENT DEVICE AND MEASUREMENT METHOD

(71) Applicant: SONY CORPORATION, Tokyo (JP)

(72) Inventors: Masaru Suzuki, Tokyo (JP); Yoshiteru Kamatani, Kanagawa (JP); Shinichiro Gomi, Tokyo (JP)

(73) Assignee: Sony Corporation, Tokyo (JP)

(21) Appl. No.: 15/123,285

(22) PCT Filed: Jan. 13, 2015
(86) PCT No.: PCT/JP2015/050621

§ 371 Date: Sep. 2, 2016

(30) Foreign Application Priority Data
Mar. 12, 2014 (JP) 2014-049503

(51) Int. Cl. A61B 5/00

(52) U.S. Cl. CPC A61B 5/0071; A61B 2562/146

(57) ABSTRACT

[Object] To measure a state of a body fluid with high accuracy in the case where a measured object is thin.

[Solution] Provided is a measurement device, including: a light source configured to emit light having a predetermined wavelength; a polarizer configured to convert the light emitted from the light source to linearly polarized light; a modulator configured to modulate a polarization direction of the linearly polarized light; at least one mirror configured to reflect the light modulated in the modulator in a measured object; an analyzer configured to separate, on the basis of a polarization direction of transmission light transmitted through the measured object, scattered light scattered in the measured object from the transmission light; and a detector configured to detect the transmission light separated from the scattered light in the analyzer.

### TECHNICAL FIELD

The present disclosure relates to a measurement device and a measurement method.

### BACKGROUND ART

In recent years, a demand for easily measuring information on an individual's physical condition without going to medical institutions has been increased because of increase in health consciousness. Specifically, a demand for easily measuring a concentration of a component and a pulsation state of an individual's body fluid (for example, blood) has been increased.

In response to such a demand, for example, Patent Literature 1 proposes a technique that, using the fact that a scattering coefficient of a biological tissue is changed in accordance with a change in glucose concentration in blood, causes near infrared light to be incident on a biological tissue and measures a scattering coefficient, thereby estimating a blood glucose level.

### CITATION LIST

### Patent Literature

Patent Literature 1: JP 2006-122579A

### SUMMARY OF INVENTION

### Technical Problem

However, in the technique disclosed in Patent Literature 1, in the case where a biological tissue serving as a measured object is so thin that a distance in which incident near infrared light passes through the measured object cannot be sufficiently secured, it is difficult to accurately measure a scattering coefficient of the biological tissue. Specifically, a measurement device using the technique disclosed in Patent Literature 1 cannot accurately measure a scattering coefficient of a thin biological tissue such as an earlobe, and therefore it is difficult to estimate a concentration of a component of a body fluid.

In view of this, the present disclosure proposes a measurement device and a measurement method, each of which is new, is improved, and is capable of measuring a state of a body fluid with high accuracy even in the case where a measured object is thin.

### Solution to Problem

According to the present disclosure, there is provided a measurement device, including: a light source configured to emit light having a predetermined wavelength; a polarizer configured to convert the light emitted from the light source to linearly polarized light; a modulator configured to modulate a polarization direction of the linearly polarized light; at least one mirror configured to reflect the light modulated in the modulator in a measured object; an analyzer configured to separate, on the basis of a polarization direction of transmission light transmitted through the measured object, scattered light scattered in the measured object from the transmission light; and a detector configured to detect the transmission light separated from the scattered light in the analyzer.

According to the present disclosure, there is provided a measurement method, including: converting light emitted from a light source configured to emit light having a predetermined wavelength to linearly polarized light; modulating a polarization direction of the linearly polarized light; reflecting the modulated light in a measured object; separating, on the basis of a polarization direction of transmission light transmitted through the measured object, scattered light scattered in the measured object from the transmission light; and detecting the transmission light separated from the scattered light.

According to the present disclosure, by reflecting light emitted for measurement in a measured object, it is possible to increase a distance in which the emitted light passes through the measured object.

### Advantageous Effects of Invention

As described above, according to the present disclosure, it is possible to measure a state of a body fluid with high accuracy even in the case where a measured object is thin.

Note that the effects described above are not necessarily limited, and along with or instead of the effects, any effect that is desired to be introduced in the present specification or other effects that can be expected from the present specification may be exhibited.

### BRIEF DESCRIPTION OF DRAWINGS

FIG. 1 is an explanatory view for describing an external appearance example of a measurement device according to an embodiment of the present disclosure.

FIG. 2 is an explanatory view for describing another external appearance example of a measurement device according to an embodiment of the present disclosure.

FIG. 3(a) is a side sectional view of a structural example of a measurement device according to an embodiment of the present disclosure, and FIG. 3(b) is a perspective view thereof.

FIG. 4 is an explanatory view of a measurement method of a measurement device according to an embodiment of the present disclosure.

FIG. 5 is a graph showing the Beer-Lambert law.

FIG. 6 is a block diagram of a functional configuration of a measurement device according to an embodiment of the present disclosure.

FIG. 7 is an explanatory view of an example of a measurement system including a measurement device according to an embodiment of the present disclosure.

FIG. 8(a) is a side sectional view of a structural example of a measurement device according to a first modification example and FIG. 8(b) is a perspective view thereof.

FIG. 9 is a side sectional view of a structural example of a measurement device according to a second modification example.

FIG. 10(a) is a side sectional view of a structural example of a measurement device according to a third modification example and FIG. 10(b) is a perspective view thereof.

FIG. 11(a) is a side sectional view of a structural example of a measurement device according to a fourth modification example and FIG. 11(b) is a perspective view thereof.

FIG. 12 is a side sectional view of a structural example of a measurement device according to a fifth modification example.

FIG. 13(a) is a side sectional view of a structural example of a measurement device according to a sixth modification example and FIG. 13(b) is a perspective view thereof.

FIG. 14(a) is a perspective view of a structural example of a measurement device according to a seventh modification example and FIG. 14(b) is a cross-sectional view thereof.

FIG. 15 is a side sectional view of a structure of a measurement device according to a comparison example.

### DESCRIPTION OF EMBODIMENT(S)

Hereinafter, (a) preferred embodiment(s) of the present disclosure will be described in detail with reference to the appended drawings. In this specification and the drawings, elements that have substantially the same function and structure are denoted with the same reference signs, and repeated explanation is omitted.

Note that description will be provided in the following order.

1. Measurement Device according to Embodiment of Present Disclosure
1.1. External Appearance Example of Measurement Device
1.2. Configuration of Measurement Device
   1.2.1. Structural Example of Measurement Device
   1.2.2. Measurement Method of Measurement Device
   1.2.3. Characteristics of Measurement Device
1.3. Functional Configuration of Measurement Device

2. Modification Examples of Measurement Device according to Embodiment of Present Disclosure
2.1. First Modification Example
2.2. Second Modification Example
2.3. Third Modification Example
2.4. Fourth Modification Example
2.5. Fifth Modification Example
2.6. Sixth Modification Example
2.7. Seventh Modification Example

3. Conclusion

### 1. MEASUREMENT DEVICE ACCORDING TO EMBODIMENT OF PRESENT DISCLOSURE

### 1.1. External Appearance Example of Measurement Device

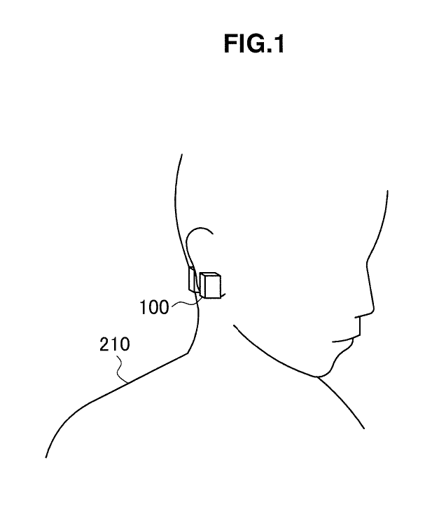

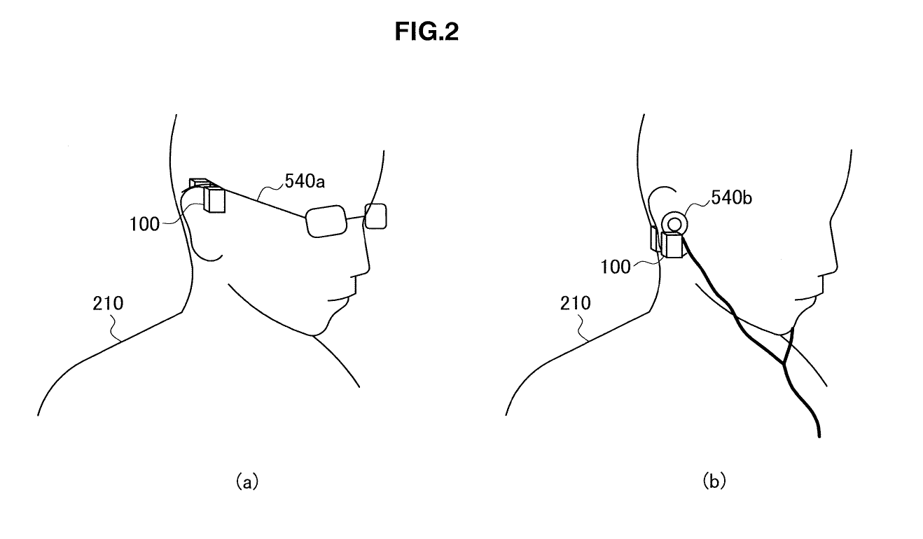

An external appearance example of a measurement device 100 according to an embodiment of the present disclosure will be described with reference to FIGS. 1 and 2. Herein, FIG. 1 is an explanatory view for describing an external appearance example of the measurement device 100 according to the embodiment of the present disclosure, and FIG. 2 is an explanatory view for describing another external appearance example of the measurement device 100 according to the embodiment of the present disclosure.

As illustrated in FIG. 1, the measurement device 100 according to the embodiment of the present disclosure is an measurement device that is attached to, for example, an earlobe of a measured subject 210 and measures a state of a body fluid of the measured subject 210.

Specifically, the measurement device 100 is a measurement device for measuring a concentration of a component of a body fluid, pulsation of the body fluid, and the like of the measured subject 210.

Herein, in order to effectively manage a physical condition of the measured subject 210, it is desirable that the measurement device for measuring a state of a body fluid of the measured subject 210 constantly or periodically measure the state of the body fluid of the measured subject 210. For example, in the case of diabetes, it is required to constantly or periodically measure a glucose concentration in blood in order to appropriately control a blood glucose level.

Therefore, in such a measurement device, in order not to impose a burden on the measured subject 210 in the case where the state of the body fluid is constantly or periodically measured, it is considered that, for example, a size of the measurement device is reduced so that the measurement device can be easily attached and measurement is performed at a terminal part of a body of the measured subject 210, such as an earlobe, a finger, a wrist, or an arm.

However, in the case where the size of the measurement device is reduced or where measurement is performed at the terminal part of the body, a biological tissue serving as a measured object is thin and therefore an amount of change in measured value is reduced, and thus it is difficult to obtain a measurement result having a sufficient accuracy.

The measurement device 100 according to the embodiment of the present disclosure has a configuration described in detail below and can therefore measure a state of a body fluid with high accuracy even in the case where a measured object is thin. Thus, the measurement device 100 according to the embodiment of the present disclosure can be reduced in size and can be easily attached to an earlobe or the like of the measured subject 210 as illustrated in FIG. 1.

As illustrated in FIG. 2, the measurement device 100 according to the embodiment of the present disclosure may be attached to the measured subject 210 in the form of a device attached to another article mounted on a living body. For example, as illustrated in FIG. 2(a), the measurement device 100 may be provided to an ear-side end portion of a temple of glasses 540a and may measure a state of a body fluid of the measured subject 210 by using an ear as a measured object. Meanwhile, as illustrated in FIG. 2(b), the measurement device 100 may be provided to a speaker portion of an earphone 540b and may measure a state of a body fluid of the measured subject 210 by using an ear as a measured object.

A part to which the measurement device 100 according to the embodiment of the present disclosure is attached is not limited to the ear illustrated in FIGS. 1 and 2. For example, the measurement device 100 may be attached to a finger, a wrist, an arm, or the like and may measure a state of a body fluid of the measured subject 210 by using the finger, the wrist, the arm, or the like as a measured object.

### 1.2. Configuration of Measurement Device

### (1.2.1. Structural Example of Measurement Device)

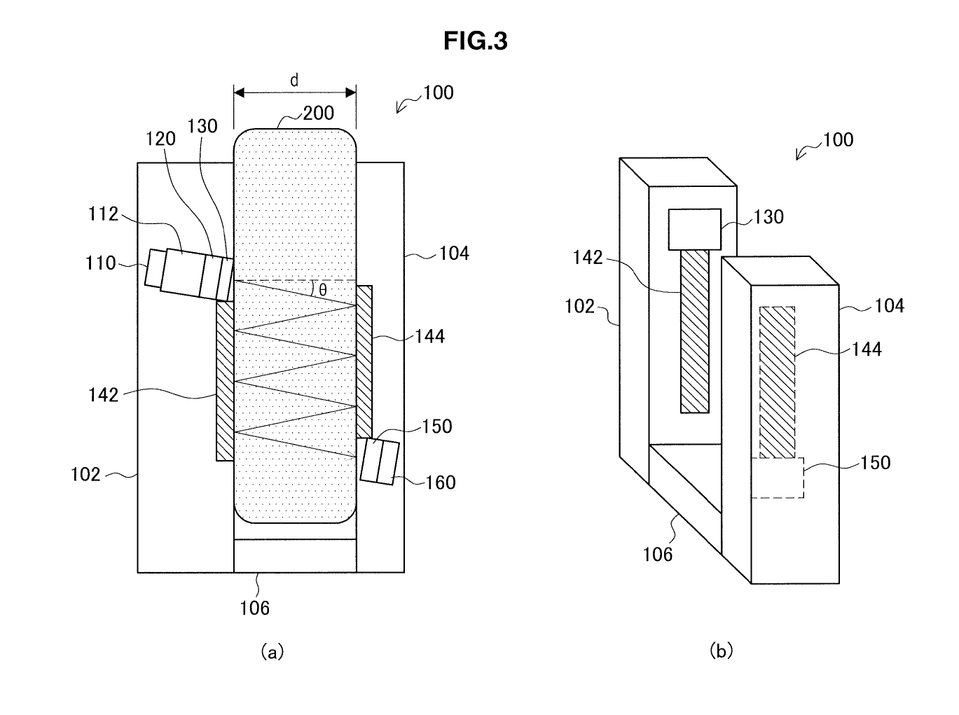

A structural example of the measurement device 100 having the above effect will be described with reference to FIG. 3. FIG. 3(a) is a side sectional view of a structural example of the measurement device 100 according to the embodiment of the present disclosure, and FIG. 3(b) is a perspective view thereof.

As illustrated in FIG. 3, the measurement device 100 includes a light source 110, a collimator 112, a the polarizer 120, a retarder 130, and a mirror 142 supported by a support member 102 and includes a mirror 144, an analyzer 150, and a detector 160 supported by a support member 104. One ends of the support members 102 and 104 are connected via a connection member 106 so that the support members 102 and 104 face each other, and a measured object 200 is sandwiched between the support members 102 and 104.

The light source 110 is a device for emitting light that has a predetermined wavelength and is emitted toward the measured object 200, and the light emitted by the light source 110 is incident on the collimator 112. The light source 110 is specifically a laser light source for performing point light emission and is more specifically a semiconductor laser. Note that, in the case where the light source 110 is a laser light source, an oscillation method is not particularly limited, and any one of a pulsed laser and a CW (Continuous Wave) laser may be used. Although a wavelength of the light emitted by the light source 110 is appropriately selected in accordance with the measured object 200, it is preferable that the wavelength be a wavelength in a near infrared region and be specifically a wavelength having about 800 nm.

The collimator 112 is provided at a subsequent stage of the light source 110 and converts the light emitted from the light source 110 to parallel rays. The light converted to the parallel rays by the collimator 112 is incident on the polarizer 120. Because the light emitted from the light source 110 is converted to the parallel rays by the collimator 112, the light can reach the detector 160 without diverging.

The polarizer 120 is provided at a subsequent stage of the collimator 112 and converts the incident light to linearly polarized light having a predetermined polarization direction. The light converted to the linearly polarized light by the polarizer 120 is incident on the retarder 130. The polarizer 120 may be, for example, a polarizing plate including a polarizing film or a prism polarizer. Note that the polarization direction of the polarizer 120 is orthogonal to a polarization direction of the analyzer 150 described below.

The retarder 130 is provided at a subsequent stage of the polarizer 120 and temporally modulates the polarization direction of the linearly polarized light converted by the polarizer 120. The light modulated by the retarder 130 is obliquely emitted at a predetermined angle with respect to a normal line of a surface of the measured object 200 which is in contact with the retarder 130. For example, the light emitted from the light source 110 toward the measured object 200 via the retarder 130 may be emitted vertically downward from a horizontal surface at an angle θ. Note that the retarder 130 may be, for example, a liquid crystal phase modulator.

The mirrors 142 and 144 are supported by the support members 102 and 104 and are provided to face each other so that the measured object 200 is sandwiched therebetween. The mirrors 142 and 144 totally reflects the light emitted from the light source 110 into the measured object 200, multiply reflects the light in the measured object 200, and then guides the light to the analyzer 150. Note that the mirrors 142 and 144 are not particularly limited as long as the mirrors can totally reflect incident light and may be, for example, mirrors or mirror-finished metal plates.

Specifically, the mirrors 142 and 144 have a substantially rectangular shape extended in a direction of a line of intersection between incident surfaces of the mirrors 142 and 144 of the light emitted into the measured object 200 and reflective surfaces of the mirrors 142 and 144 thereof. For example, as illustrated in FIG. 3, in the case where light is emitted from the light source 110 toward the measured object 200 in an upward or downward direction of a direction vertical to the horizontal surface, the mirrors 142 and 144 may have a rectangular shape extended in the direction vertical to the horizontal surface. Meanwhile, in the case where light is emitted from the light source 110 toward the measured object 200 in a leftward or rightward direction of a horizontal direction, the mirrors 142 and 144 may have a rectangular shape extended in the horizontal direction.

Note that, although FIG. 3 illustrates a configuration in which the measurement device 100 includes the pair of mirrors 142 and 144, the measurement device 100 according to the embodiment of the present disclosure is not limited to the example illustrated in FIG. 3. As described in a first modification example below, the measurement device 100 according to the embodiment of the present disclosure only needs to include at least one mirror.

The analyzer 150 is provided at a preceding stage of the detector 160 and allows only light that has been transmitted through the measured object 200 and has a polarization direction vertical to the polarization direction of the polarizer 120 to pass therethrough. The light transmitted through the analyzer 150 is received by the detector 160. Specifically, the analyzer 150 is, for example, a polarizing plate having a polarization direction orthogonal to the polarization direction of the polarizer 120 and may be, for example, a polarizing plate including a polarizing film or a prism polarizer.

The detector 160 is placed at a position at which the light that has been multiply reflected by the mirrors 142 and 144 in the measured object 200 and has been transmitted therethrough is receivable and converts the received light to electrical signals and thus detects the electrical signals. For example, the detector 160 may be a photomultiplier tube or photodiode for generating a current on the basis of an intensity of the received light.

The support members 102 and 104 are substantially rectangular parallelepiped members facing each other so that the measured object 200 is sandwiched therebetween, and the one ends of the support members 102 and 104 are connected by the connection member 106. The support member 102 supports the light source 110, the collimator 112, the polarizer 120, the retarder 130, and the mirror 142, and the support member 104 supports the mirror 144, the analyzer 150, and the detector 160. The support members 102 and 104 are preferably made of a light absorbing material in order to prevent stray light.

The connection member 106 is a substantially rectangular parallelepiped member that connects the one ends of the support members 102 and 104. The connection member 106 is provided so that a distance d between the support members 102 and 104 is changeable. For example, the connection member 106 may be provided to be insertable into the support members 102 and 104, and the support members 102 and 104 may be provided to be slidably movable along the connection member 106. In such a case, positions of the respective support members 102 and 104 are fixed by fixing the support members 102 and 104 to the connection member 106 with springs, screws, or the like. With this configuration, the distance d between the support members 102 and 104 can be appropriately changed, and therefore the measurement device 100 can measure the measured objects 200 having various thicknesses while the measured object 200 being sandwiched.

The measured object 200 is, for example, a living body, and, more specifically, encompasses terminal parts of a body of the measured subject 210, such as an earlobe, a finger, a wrist, and an arm. The measured object 200 is sandwiched between the support members 102 and 104, and a state of a body fluid thereinside is measured by transmitting light emitted from the light source 110 through the measured object 200 and detecting the light with the use of the detector 160.

### 1.2.2. Measurement Method of Measurement Device

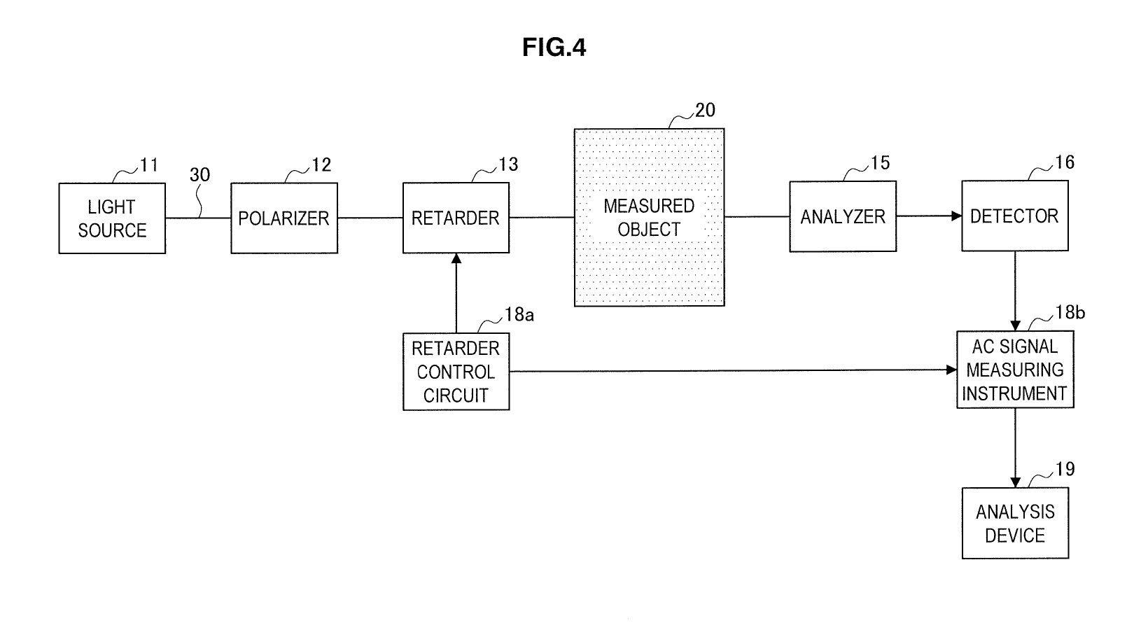

A method of measuring a state of a body fluid in the measurement device 100 according to the embodiment of the present disclosure will be described with reference to FIG. 4. FIG. 4 is an explanatory view of a measurement method of the measurement device 100 according to the embodiment of the present disclosure.

The measurement device 100 according to the embodiment of the present disclosure is, for example, a device for measuring a concentration of a component in a body fluid by using the fact that a scattering coefficient of a body fluid contained in a biological tissue is changed in accordance with a change in concentration of a component in the body fluid. Specifically, the scattering coefficient in the biological tissue depends on a difference between a refractive index of a minute biological material (for example, red blood cells, white blood cells, platelets, or a cell membrane) which is a scatterer and a refractive index of the body fluid which is a medium. It is known that the refractive index of the minute biological material is larger than the refractive index of the body fluid. Thus, in the case where the concentration of the component in the body fluid is increased, the refractive index of the body fluid is increased and the difference between the refractive index of the minute biological material and the refractive index of the body fluid is reduced. Therefore, the concentration of the component in the body fluid can be measured by measuring the scattering coefficient of the biological tissue with respect to light transmitted through the biological tissue.

However, in the above method, the biological tissue has a high scattering coefficient, and therefore there is a possibility that not only straight light that has moved straight and has been transmitted through the biological tissue without scattering, but also scattered light that has been multiply scattered in the biological tissue and has moved around reaches the detector. Therefore, the measurement device 100 according to the embodiment of the present disclosure separates the scattered light that has been multiply scattered in the biological tissue and has moved around to reach the detector by using the method described below with reference to FIG. 4.

As shown in FIG. 4, light 30 emitted from a light source 11 passes through a polarizer 12, a retarder 13, a measured object 20, and an analyzer 15, thereby entering a detector 16. The retarder 13 is controlled by a retarder control circuit 18a, and a result detected in the detector 16 is analyzed by an analysis device 19 via an AC signal measuring instrument 18b.

Herein, in FIG. 4, the light source 11 corresponds to the light source 110, the polarizer 12 corresponds to the polarizer 120, the retarder 13 corresponds to the retarder 130, the measured object 20 corresponds to the measured object 200, the analyzer 15 corresponds to the analyzer 150, and the detector 16 corresponds to the detector 160. The retarder control circuit 18a, the AC signal measuring instrument 18b, and the analysis device 19 are a control circuit and an arithmetic processing circuit which are not illustrated in FIG. 3. Note that the AC signal measuring instrument 18b is, for example, a lock-in amplifier.

First, light emitted from the light source 11 is converted to linearly polarized light (for example, light having a vertical polarization direction) by the polarizer 12. Then, the light emitted from the light source 11 is modulated by the retarder 13 to light obtained by temporally changing a polarization direction of the linearly polarized light (for example, light having a vertical polarization direction and a horizontal polarization direction alternately).

The light modulated by the retarder 13 is emitted toward the measured object 20. Herein, regarding straight light that has been transmitted through the measured object 20 without scattering therein, a polarization direction thereof obtained when the straight light is incident thereon is maintained. Meanwhile, regarding scattered light that has been multiply scattered and has moved around, a polarization direction thereof obtained when the scattered light is incident thereon is not maintained, and the scattered light becomes light oscillating in various directions. That is, the straight light that has been transmitted through the measured object 20 without scattering therein can be detected as light whose polarization direction has been temporally changed, and the scattered light that has been multiply scattered and has moved around can be detected as light whose polarization direction has not been temporally changed.

In view of this, when the light that has been transmitted through the measured object 20 passes through the analyzer 15 having a polarization direction (for example, horizontal direction) vertical to a polarization direction of the polarizer 12 and is then detected by the detector 16, it is possible to separate the straight light that has been transmitted without scattering, which serves as an AC component, from the scattered light that has been multiply scattered and has moved around, which serves as a DC component.

Then, when an output signal from the detector 16 is synchronously detected by the AC signal measuring instrument 18b synchronized with a drive signal of the retarder control circuit 18a for controlling the retarder 13, it is possible to acquire an output signal of the light that has been transmitted without scattering. Note that the drive signal for controlling the retarder 13 may be output to the retarder 13 from the AC signal measuring instrument 18b, and, in such a case, the retarder control circuit 18a is included in the AC signal measuring instrument 18b.

Further, when the acquired output signal is analyzed in the analysis device 19, it is possible to analyze a change in scattering coefficient of the measured object 20 and to measure a concentration of a component of a body fluid of the measured object 20. The DC component detected by the detector 16 has a value depending on pulsation of the body fluid, and therefore, when the DC component of the light detected by the detector 16 is analyzed in the analysis device 19, a pulsation state of the body fluid can also be measured.

With the measurement method described above, the measurement device 100 according to the embodiment of the present disclosure can measure a state of a body fluid of the measured object 20.

### 1.2.3. Characteristics of Measurement Device

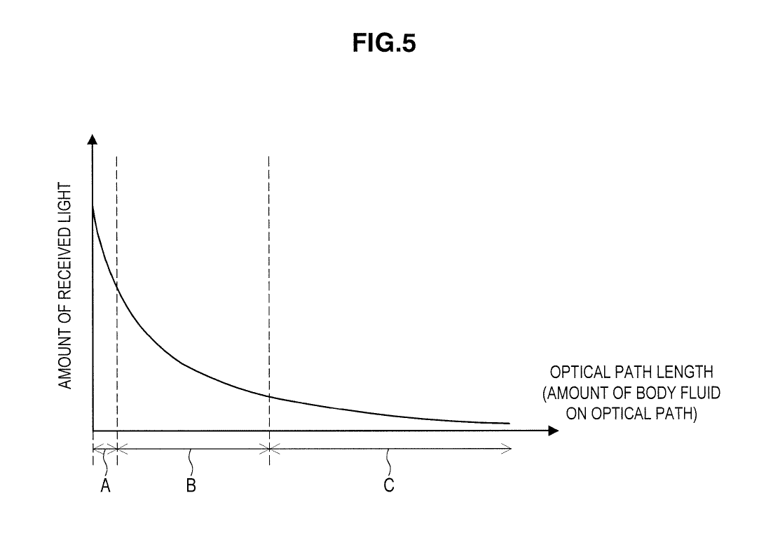

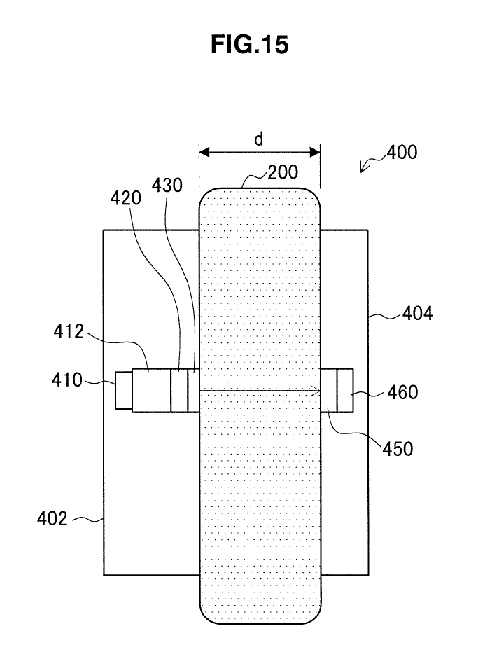

Characteristics of the measurement device 100 according to the embodiment of the present disclosure will be described with reference to FIGS. 5 and 15 by comparing the measurement device 100 with a measurement device 400 according to a comparison example. FIG. 5 is a graph showing the Beer-Lambert law, and FIG. 15 is a side sectional view of a structure of the measurement device 400 according to the comparison example.

A structure of the measurement device 400 according to the comparison example will be described with reference to FIG. 15. As illustrated in FIG. 15, in the measurement device 400 according to the comparison example, a light source 410 corresponds to the light source 110 in FIG. 3, a collimator 412 corresponds to the collimator 112 in FIG. 3, a polarizer 420 corresponds to the polarizer 120 in FIG. 3, a retarder 430 corresponds to the retarder 130 in FIG. 3, an analyzer 450 corresponds to the analyzer 150 in FIG. 3, a detector 460 corresponds to the detector 160 in FIG. 3, a support member 402 corresponds to the support member 102 in FIG. 3, and a support member 404 corresponds to the support member 104 in FIG. 3. That is, the measurement device 400 illustrated in FIG. 15 is different from the measurement device 100 illustrated in FIG. 3 in that the measurement device 400 does not include a configuration corresponding to the mirrors 142 and 144 and light incident on the measured object 200 is directly incident on the detector 460.

Herein, when an optical path length obtained in the case where light is incident on the measured object 200 having the same width d is calculated, the optical path length is “d” in the measurement device 400 illustrated in FIG. 15 according to the comparison example, whereas, in the measurement device 100 illustrated in FIG. 3 according to the embodiment of the present disclosure, the optical path length is expressed by the following numerical expression 1.

$$\begin{matrix}[ Math . 1 ] & \\ \frac{( N + 1 ) * d}{cos θ} & Numerical expression 1\end{matrix}$$

In the numerical expression 1, N denotes the number of times of reflection by the mirrors 142 and 144, and θ denotes an angle between an emission direction of light emitted toward the measured object 200 and a normal line to a surface of the measured object 200 on which the light is incident.

Herein, θ falls within the range of 0°<θ<90°, and therefore 0<cos θ<1 is satisfied. Therefore, it is found that “d/cos θ” is larger than “d”. In the measurement device 100 according to the embodiment of the present disclosure, the optical path length is further increased for a length corresponding to the number of times of reflection N by the mirrors 142 and 144.

Therefore, as compared with the measurement device 400 according to the comparison example, the measurement device 100 according to the embodiment of the present disclosure further includes the mirrors 142 and 144 and can remarkably increase the optical path length in the measured object 200 by causing light incident on the measured object 200 to be reflected by the mirrors 142 and 144. Therefore, even in the case where the measured object 200 is thin, the measurement device 100 according to the embodiment of the present disclosure can increase the optical path length by reflection and can therefore measure a state of a body fluid with high accuracy. Note that the number of times of reflection by the mirrors 142 and 144 in the measured object 200 can be set to an arbitrary number of times.

However, as shown in FIG. 5, according to the Beer-Lambert law, in the case where an amount of incident light is constant, an amount of received light received by the detector 160 is logarithmically reduced as the optical path length is increased (that is, as an amount of a body fluid on an optical path is increased). Therefore, for example, in the case of an optical path length within a range of “C” in FIG. 5, the optical path length in the measured object 200 is long and absorption into the measured object 200 is increased according to the Beer-Lambert law, and therefore the amount of received light received by the detector 160 is reduced. Thus, measurement accuracy is reduced, which is not preferable. Meanwhile, in the case of an optical path length within a range of “A” in FIG. 5, the optical path length in the measured object 200 is short and an amount of change in concentration of a component of a body fluid is small. Thus, measurement accuracy is reduced, which is not preferable.

Therefore, it is preferable that the measurement device 100 according to the embodiment of the present disclosure control an incident angle of light on the measured object 200 and the number of times of reflection of the light so as to achieve an optical path length within a range of “B” in FIG. 5 in which the amount of body fluid on the optical path is not too small and the amount of received light is not excessively reduced. Herein, the optical path length within the range of “B” in FIG. 5 is, for example, 5 mm or more but 20 mm or less.

### 1.3. Functional Configuration of Measurement Device

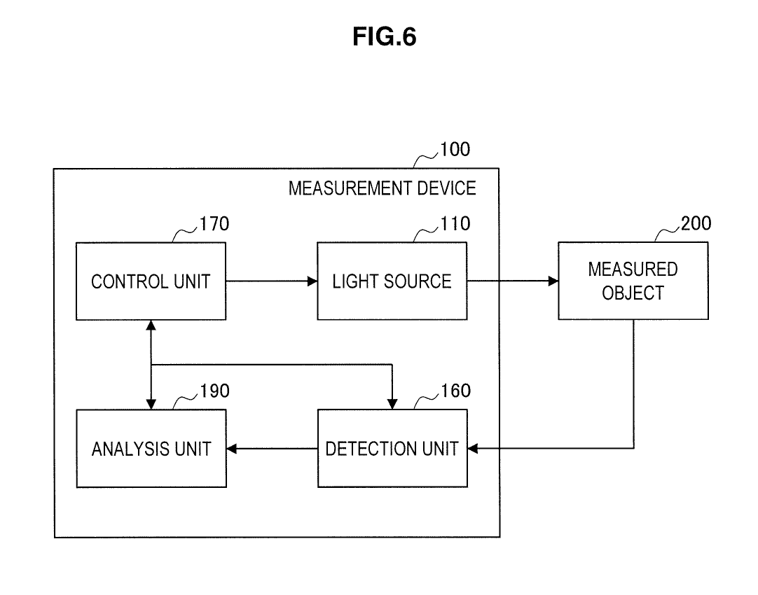

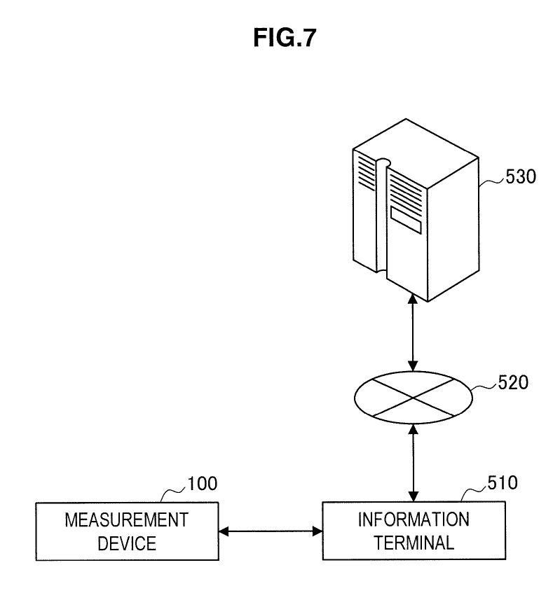

A functional configuration of the measurement device 100 according to the embodiment of the present disclosure will be described with reference to FIGS. 6 and 7. FIG. 6 is a block diagram of a functional configuration of the measurement device 100 according to the embodiment of the present disclosure, and FIG. 7 is an explanatory view of an example of a measurement system including the measurement device 100 according to the embodiment of the present disclosure.

As shown in FIG. 6, the measurement device 100 according to the embodiment of the present disclosure includes the light source 110, a detection unit 160, a control unit 170, and an analysis unit 190. The measurement device 100 measures a state of a body fluid in the measured object 200 by emitting light from the light source 110 toward the measured object 200 and detecting the light transmitted through the measured object 200 with the use of the detection unit 160.

Herein, the light source 110 is substantially equal to the light source 110 described with reference to FIG. 3, the detection unit 160 is substantially equal to the detection unit 160 described with reference to FIG. 3, and the measured object 200 is substantially equal to the measured object 200 described with reference to FIG. 3. Therefore, description thereof is herein omitted.

The control unit 170 controls each configuration (for example, light source 110) of the measurement device 100 in order to allow the measurement device 100 to measure the measured object 200. Specifically, the control unit 170 may determine the thickness of the measured object 200 on the basis of the distance d between the support members 102 and 104 which is changeable by the connection member 106 and may control output of the light source 110 in accordance with the determined thickness of the measured object 200.

Further, the control unit 170 may control output of the light source 110 on the basis of the amount of received light detected by the detector 160. Specifically, the control unit 170 may perform control to increase output of the light source 110 in the case where the control unit 170 determines that the amount of received light detected by the detector 160 is not sufficient to measure a state of a body fluid. In the case where, although the light source 110 emits light, the detector 160 does not detect the light, the control unit 170 may determine that abnormality has occurred and stop light emission of the light source 110. With this configuration, the control unit 170 can control the light source 110 so that the light source 110 performs optimal output for measurement and can stop light emission of the light source 110 in the case where measurement is not performed. This makes it possible to reduce power consumption. Further, the control unit 170 can prevent light emitted from the light source 110 from leaking to the surrounding area in the case where measurement is not performed.

Furthermore, the control unit 170 may perform control so that the measurement device 100 measures a state of a body fluid at a predetermined cycle and may issue warning to the measured subject 210 in the case where a measurement result has an abnormal value. Specifically, in the case where the measurement result has an abnormal value, there is a possibility that the measurement device 100 is almost detached from an attached part or a surface state is changed due to sweat or the like. Therefore, it is preferable that the control unit 170 issue a warning that abnormality has occurred in measurement to the measured subject 210 with sound, light, or the like and encourage the measured subject 210 to reattach the measurement device 100. Note that, in the case where the control unit 170 controls warning operation, the control unit 170 may additionally display information indicating an abnormal state. For example, the control unit 170 may display an abnormal value together with the warning. Herein, regarding determination on whether or not a value is abnormal in the control unit 170, for example, when a normal value range is set in advance, the control unit 170 may determine that a value beyond the normal value range is an abnormal value, or the control unit 170 may determine that a value largely deviating from an average of measurement results is an abnormal value. With this configuration, in the case where a state of a body fluid is measured at a predetermined cycle, the control unit 170 can promptly notify the measured subject 210 of abnormality of measurement.

Note that, although description will be made in modification examples described below, in the case where an arrangement position and a direction of any one of the mirrors 142 and 144, the light source 110, and the detector 160 are changeable, the control unit 170 may perform control so that the arrangement positions and the directions of the mirrors 142 and 144, the light source 110, and the detector 160 are optimized for measurement.

The analysis unit 190 analyzes a state of a body fluid on the basis of the amount of received light detected by the detection unit 160. Specifically, the analysis unit 190 acquires the distance d between the support members 102 and 104 and the angle θ between an emission direction of light emitted toward the measured object 200 and a normal line to the surface of the measured object 200 on which the light is incident and calculates the number of times of reflection N, thereby calculating an optical path length in the measured object 200. Then, the analysis unit 190 compares an intensity of the light incident on the measured object 200 with an intensity of straight light transmitted through the measured object 200 without scattering, calculates a scattering amount of the measured object 200, and calculates a scattering coefficient on the basis of the optical path length. Further, the analysis unit 190 has a standard curve of a concentration of a component with respect to a scattering coefficient for each component of the body fluid and calculates the concentration of the component of the body fluid on the basis of the standard curve by using the calculated scattering coefficient.

Note that the measurement device 100 may further include a sensor for measuring a state of the measured object 200 or a state of an environment in which the measured object 200 is placed. In such a case, the analysis unit 190 may correct the concentration of the component of the body fluid calculated with the above method on the basis of a result of measurement performed by the sensor. Specifically, in the case where the measurement device 100 includes a clinical thermometer, the analysis unit 190 may correct and calculate, on the basis of a body temperature of the measured subject 210, the concentration of the component of the body fluid calculated by using the scattering coefficient.

Herein, the control unit 170 and the analysis unit 190 may be configured by hardware such as a central processing unit (CPU), a read only memory (ROM), and a random access memory (RAM). Specifically, the CPU may function as an arithmetic processing unit and a control device and execute control performed by the control unit 170 and the analysis unit 190 in accordance with various programs. The ROM may store the programs used by the CPU and operation parameters, and the RAM may temporarily store programs for use in execution of the CPU, parameters that appropriately change in the execution thereof, and the like.

A measurement system including the above measurement device 100 according to the embodiment of the present disclosure will be described. As illustrated in FIG. 7, the measurement device 100 according to the embodiment of the present disclosure may further include a communication unit that can communicate with external apparatuses, may be connected to an information terminal 510, and may further be connected to an external server 530 via a public network 520.

The information terminal 510 communicates, for example, with the measurement device 100, receives a measurement result measured by the measurement device 100, and displays the measurement result. Further, the information terminal 510 may receive a warning that a measured value from the measurement device 100 is an abnormal value and may display the warning. Furthermore, the information terminal 510 communicates with the external server 530 via the public network 520 and transmits the measurement result measured by the measurement device 100 to the external server 530.

The public network 520 is, for example, a public network such as the Internet, a satellite communication network, or a telephone network, a local area network (LAN), or a wide area network (WAN).

The external server 530 analyzes the measurement result measured by the measurement device 100 and then stores an analysis result thereof. For example, the external server 530 chronologically stores a measurement result of a state of a body fluid measured by the measurement device 100, analyzes a chronological change thereof, and stores an analysis result thereof. Further, the external server 530 transmits the stored analysis result to the information terminal 510 in response to a request of the information terminal 510.

According to the above measurement system including the measurement device 100 according to the embodiment of the present disclosure, the measured subject 210 can store measurement results of the state of the body fluid measured by the measurement device 100 in the external apparatuses such as the information terminal 510 and the external server 530. Further, the measured subject 210 can chronologically arrange and analyze the stored measurement results with the use of the external server 530 and the like, and therefore it is possible to analyze the state of the body fluid in more detail.

Note that, although the measurement device 100 including the control unit 170 and the analysis unit 190 has been described hereinabove, a part or all of the control unit 170 and the analysis unit 190 may be included in the information terminal 510 or the external server 530. In such a case, for example, the measurement device 100 detects light transmitted through the measured object 200, and thereafter calculation of a scattering coefficient and analysis of a state of a body fluid are performed by the information terminal 510 or the external server 530.

Hereinabove, the measurement device 100 according to the embodiment of the present disclosure has been described in detail.

### 2. MODIFICATION EXAMPLES OF MEASUREMENT DEVICE ACCORDING TO EMBODIMENT OF PRESENT DISCLOSURE

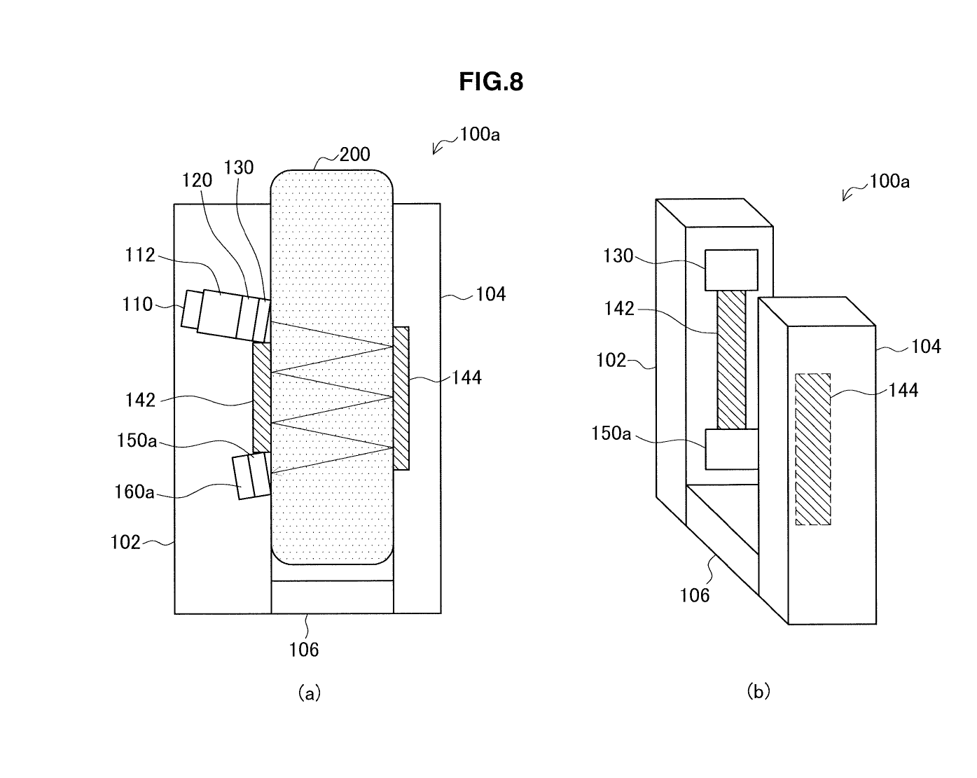

The first to seventh modification examples of the measurement device according to the embodiment of the present disclosure will be described with reference to FIGS. 8 to 14. Note that the first to seventh modification examples described below can be combined with one another as long as there is no inconsistency between configurations thereof, and those combinations also fall within the technical scope of the present disclosure. Hereinbelow, differences between measurement devices according to the first to seventh modification examples and the measurement device 100 illustrated in FIG. 3 according to the embodiment of the present disclosure will be mainly described, and description of substantially similar configurations is omitted.

### 2.1. First Modification Example

The first modification example of the measurement device according to the embodiment of the present disclosure will be described with reference to FIG. 8. FIG. 8(a) is a side sectional view of a structural example of a measurement device 100a according to the first modification example and FIG. 8(b) is a perspective view thereof.

As illustrated in FIG. 8, the measurement device 100a according to the first modification example is different from the measurement device 100 illustrated in FIG. 3 in that an analyzer 150a and a detector 160a are supported by the same support member 102 that supports the light source 110, the collimator 112, the polarizer 120, and the retarder 130. In such a case, the mirror 142 is placed to be inserted between the light source 110, the collimator 112, the polarizer 120, and the retarder 130 and the analyzer 150a and the detector 160a, and the mirror 142 has a substantially rectangular shape extended in a direction of a straight line connecting the retarder 130 and the analyzer 150a.

Also with this configuration, the measurement device 100a, as well as the measurement device 100 illustrated in FIG. 3, can reflect light emitted from the light source 110 toward the measured object 200 via the retarder 130 in the measured object 200 and detect the light with the use of the detector 160a.

In the above first modification example, the measurement device 100a only needs to include at least one mirror for reflecting light emitted toward the measured object 200. Specifically, the detector 160a may receive and detect light that has been emitted from the light source 110 toward the measured object 200 and has been reflected by the mirror 144 once.

### 2.2. Second Modification Example

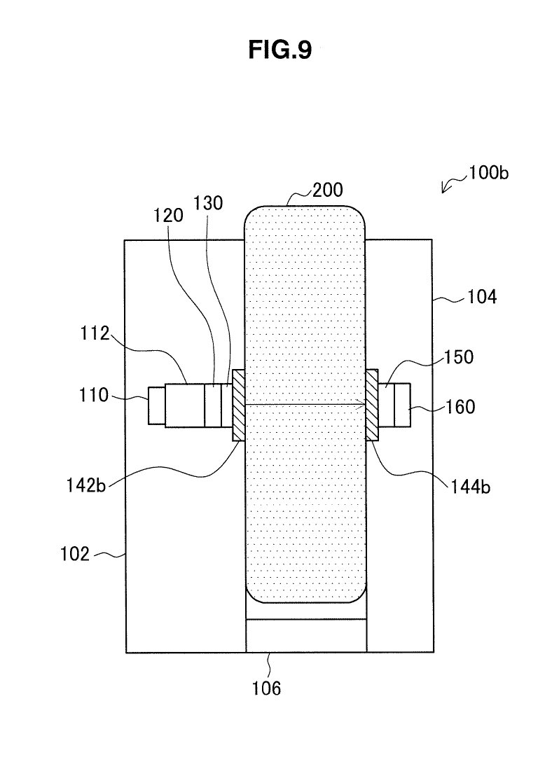

A second modification example of the measurement device according to the embodiment of the present disclosure will be described with reference to FIG. 9. FIG. 9 is a side sectional view of a structural example of a measurement device 100b according to the second modification example.

As illustrated in FIG. 9, the measurement device 100b according to the second modification example is different from the measurement device 100 illustrated in FIG. 3 in that mirrors 142b and 144b are made up of half mirrors, and the mirror 142b is provided at a subsequent stage of the retarder 130, whereas the mirror 144b is provided at a preceding stage of the analyzer 150. In the measurement device 100b according to the second modification example, the light source 110, the collimator 112, the polarizer 120, and the retarder 130 and the analyzer 150 and the detector 160 are placed to face each other.

In such a case, light emitted from the light source 110 toward the measured object 200 via the retarder 130 is partially reflected by the mirrors 142b and 144b which are half mirrors and repeatedly reflected between the mirrors 142b and 144b. Herein, the analysis unit 190 separates light that has been reflected a predetermined number of times to have an optical path length set in advance from light detected by the detector 160 on the basis of a phase or the like, thereby calculating a scattering coefficient of the measured object 200. Also with this configuration, the measurement device 100b, as well as the measurement device 100 illustrated in FIG. 3, can calculate a scattering coefficient of the measured object 200 and can measure a state of a body fluid of the measured object 200.

### 2.3. Third Modification Example

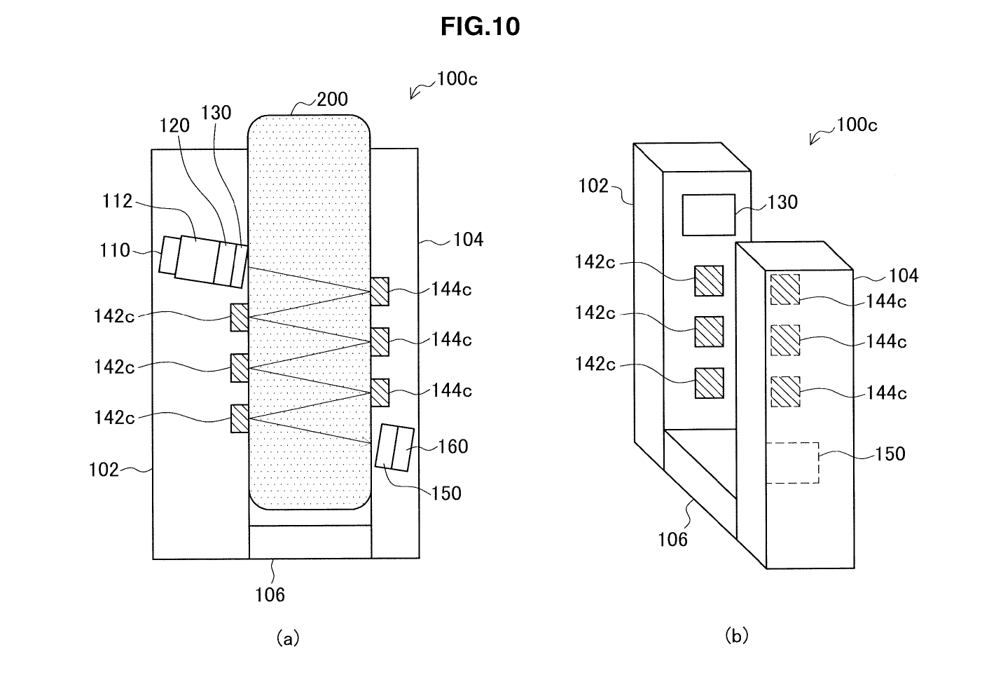

The third modification example of the measurement device according to the embodiment of the present disclosure will be described with reference to FIG. 10. FIG. 10(a) is a side sectional view of a structural example of a measurement device 100c according to the third modification example and FIG. 10(b) is a perspective view thereof.

As illustrated in FIG. 10, the measurement device 100c according to the third modification example is different from the measurement device 100 illustrated in FIG. 3 in that a plurality of mirrors 142c and 144c are placed instead of the mirrors 142 and 144. The plurality of mirrors 142c and 144c are provided in accordance with reflection positions of light in the measured object 200 and reflect light on an optical axis of light emitted into the measured object 200. For example, in the case where light is emitted from the light source 110 toward the measured object 200 in the downward direction of the direction vertical to the horizontal surface, the mirrors 142c and 144c may be a plurality of mirrors divided in the direction vertical to the horizontal surface and may be placed at positions corresponding to reflection positions of the light.

With this configuration, only the light on the optical axis of the light emitted into the measured object 200 is reflected by the mirrors 142c and 144c, and therefore light that has been scattered by the measured object 200 and has deviated from the optical axis is not reflected by the mirrors 142c and 144c. Thus, the measurement device 100c can prevent the scattered light that has been scattered by the measured object 200 and has deviated from the optical axis from entering the detector 160 and can guide, to the detector 160, only straight light transmitted through the measured object 200 without scattering therein.

Each of the mirrors 142c and 144c may be an independent movable mirror whose at least one of an arrangement position and a direction is changeable. In such a case, an actuator or the like is provided in each of the mirrors 142c and 144c, and the arrangement position and the direction of each of the mirrors 142c and 144c are optimized by the control unit 170 so that light emitted into the measured object 200 is guided to the detector 160. Specifically, the arrangement position and the direction of each of the mirrors 142c and 144c are controlled so that an intensity of a signal detected by the detector 160 is maximized. Note that the actuators included in the mirrors 142c and 144c may be, for example, an electromagnetic conversion actuator, a piezoelectric actuator, an electrostatic actuator, a shape memory alloy (SMA) actuator, or an electroactive polymer (EAP) actuator. The arrangement position and the direction of each of the mirrors 142c and 144c controlled by the control unit 170 are transmitted to the analysis unit 190 in order to calculate an optical path length of light emitted into the measured object 200.

With this configuration, the measurement device 100c can change the number of times of reflection of light emitted into the measured object 200 by changing the arrangement position and the direction of each of the mirrors 142c and 144c. Therefore, the measurement device 100c can change the optical path length in the measured object 200 more flexibly in accordance with the distance d between the support members 102 and 104, the angle θ between an emission direction of light emitted toward the measured object 200 and a normal line to the surface of the measured object 200 on which the light is incident, the amount of received light detected by the detector 160, and the like.

Each of the support members 102 and 104 is an elastic member or a member including a movable portion, and a shape thereof may be changed in accordance with a surface shape of the measured object 200. For example, the support members 102 and 104 may be made of elastic resin whose shape is reversibly changeable (for example, urethane resin). Also in the case where the shapes of the support members 102 and 104 are changed as described above, each of the mirrors 142c and 144c can be optimized by changing the arrangement position and the direction with the use of the control unit 170 so that light emitted into the measured object 200 is guided to the detector 160.

With this configuration, the measurement device 100c can cause the shapes of the support members 102 and 104 to fit a biological tissue serving as the measured object 200 and can bring the support members 102 and 104 into close contact with the biological tissue, and therefore the measurement device 100c can acquire a more accurate optical path length and perform measurement.

### 2.4. Fourth Modification Example

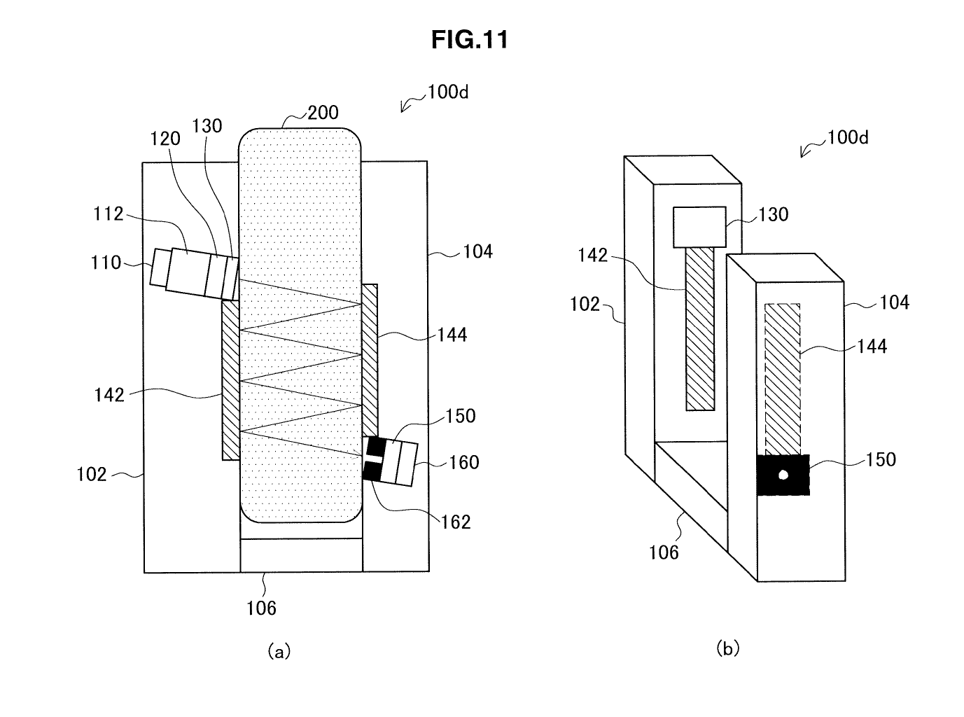

The fourth modification example of the measurement device according to the embodiment of the present disclosure will be described with reference to FIG. 11. FIG. 11(a) is a side sectional view of a structural example of a measurement device 100d according to the fourth modification example and FIG. 11(b) is a perspective view thereof.

As illustrate in FIG. 11, the measurement device 100d according to the fourth modification example is different from the measurement device 100 illustrated in FIG. 3 in that a pinhole 162 is provided at a preceding stage of the analyzer 150. The pinhole 162 is a light-absorbent member in which a hole having an arbitrary size is formed at a position corresponding to an optical axis of light emitted into the measured object 200. The pinhole 162 allows straight light on the optical axis of the light emitted into the measured object 200 to pass through the hole and absorbs scattered light that has been scattered by the measured object 200 and has deviated from the optical axis.

With this configuration, the measurement device 100d can prevent the scattered light that has been scattered by the measured object 200 and has deviated from the optical axis from entering the detector 160 and can guide, to the detector 160, only the straight light that has been transmitted through the measured object 200 without scattering therein.

Note that an arrangement position of the pinhole 162 is not limited to the preceding stage of the analyzer 150. For example, the pinhole 162 may be provided at a preceding stage of the detector 160 or may be provided at both the preceding stages of the analyzer 150 and the detector 160.

### 2.5. Fifth Modification Example

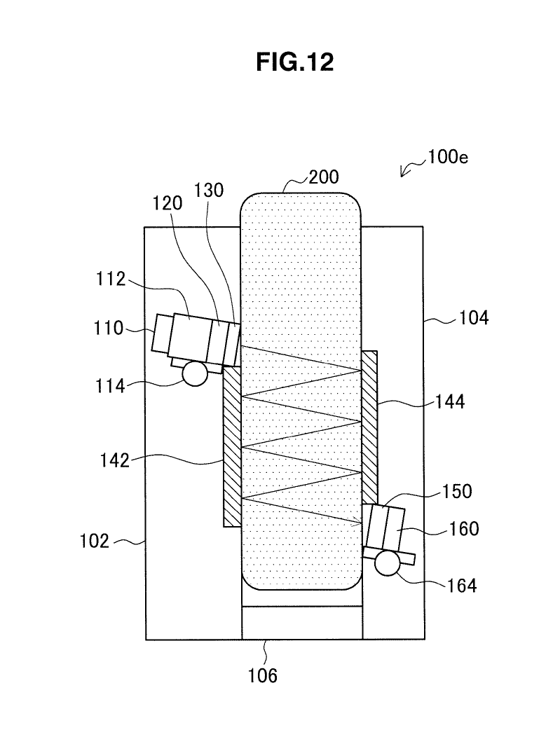

The fifth modification example of the measurement device according to the embodiment of the present disclosure will be described with reference to FIG. 12. FIG. 12 is a side sectional view of a structural example of a measurement device 100e according to the fifth modification example.

As illustrated in FIG. 12, the measurement device 100e according to the fifth modification example is different from the measurement device 100 illustrated in FIG. 3 in that the measurement device 100e further includes a light source rocking mechanism 114 and a detector rocking mechanism 164. Note that the measurement device 100e may include at least one of the light source rocking mechanism 114 and the detector rocking mechanism 164.

The light source rocking mechanism 114 supports the light source 110, the collimator 112, the polarizer 120, and the retarder 130 and rocks those supported configurations with the use of an actuator or the like. The detector rocking mechanism 164 supports the analyzer 150 and the detector 160 and rocks those supported configurations with the use of an actuator or the like. Note that the actuators included in the light source rocking mechanism 114 and the detector rocking mechanism 164 may be, for example, an electromagnetic conversion actuator, a piezoelectric actuator, an electrostatic actuator, a shape memory alloy (SMA) actuator, or an electroactive polymer (EAP) actuator.

With this configuration, the measurement device 100e can cause the control unit 170 to independently rock the light source rocking mechanism 114 and the detector rocking mechanism 164 and can optimize the arrangement positions and the directions of the light source 110, the collimator 112, the polarizer 120, the retarder 130, the analyzer 150, and the detector 160 so that an intensity of a signal detected by the detector 160 is maximized. Note that the measurement device 100e may perform the above optimization using the light source rocking mechanism 114 and the detector rocking mechanism 164 when the measurement device 100e is attached to the measured subject 210, may perform the above optimization when measurement is started, or may perform the above optimization at any time.

### 2.6. Sixth Modification Example

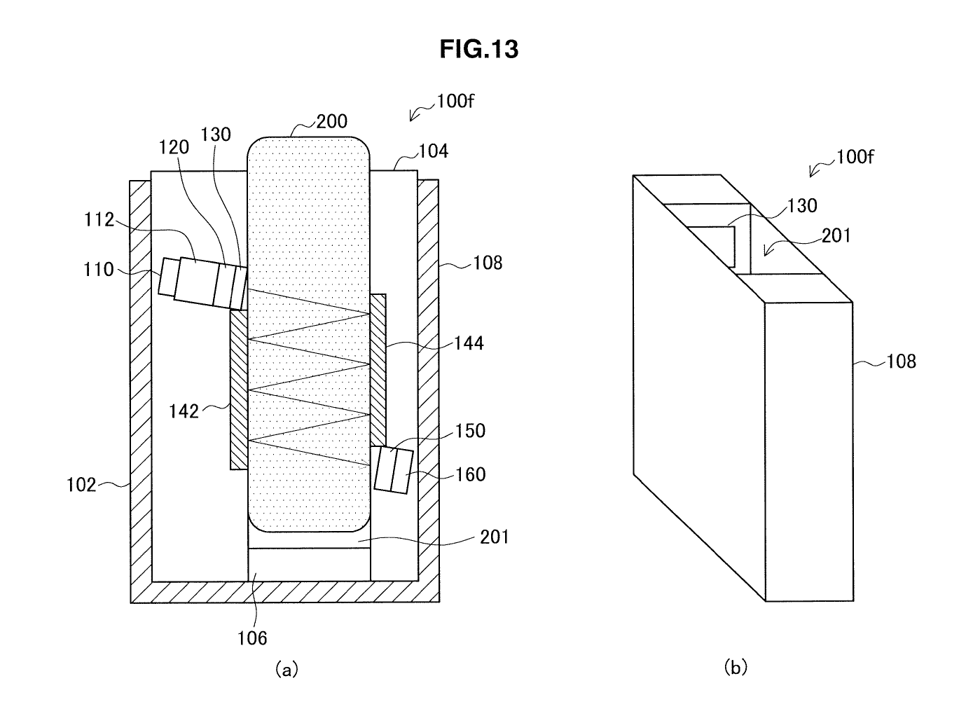

The sixth modification example of the measurement device according to the embodiment of the present disclosure will be described with reference to FIG. 13. FIG. 13(a) is a side sectional view of a structural example of a measurement device 100f according to the sixth modification example and FIG. 13(b) is a perspective view thereof.

As illustrated in FIG. 13, the measurement device 100f according to the sixth modification example is different from the measurement device 100 illustrated in FIG. 3 in that the measurement device 100f further includes a covering member 108. The covering member 108 is a light-absorbent member for covering a measurement region 201 into which the measured object 200 is inserted and light is emitted from the light source 110. Specifically, the covering member 108 covers the measurement region 201 surrounded by a U shape formed by the support members 102 and 104 and the connection member 106. For example, the covering member 108 may cover all side surfaces and a bottom surface of the measurement device 100f. Note that, also in such a case, the connection member 106 can change the distance d between the support members 102 and 104.

With this configuration, the covering member 108 can prevent light emitted from the light source 110 from leaking to the outside of the measurement region 201. The covering member 108 can also prevent accuracy of a measurement result from being reduced due to entering of natural light into the measurement region 201. Therefore, according to the measurement device 100f, it is possible to improve safety at the time of measurement and to prevent reduction in accuracy of a measurement result.

### 2.7. Seventh Modification Example

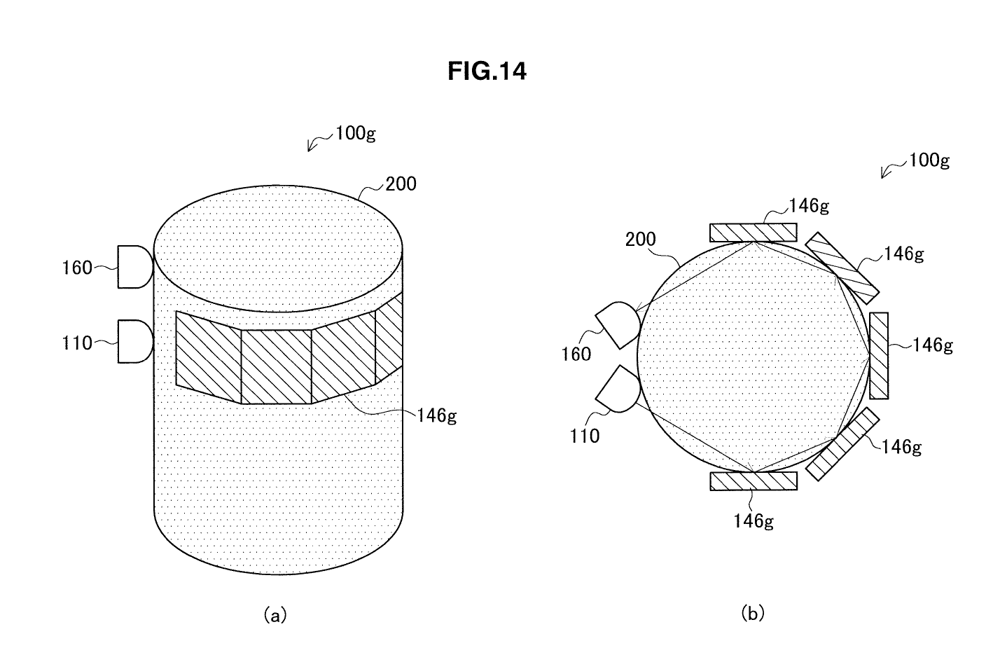

The seventh modification example of the measurement device according to the embodiment of the present disclosure will be described with reference to FIG. 14. FIG. 14(a) is a perspective view of a structural example of a measurement device 100g according to the seventh modification example and FIG. 14(b) is a cross-sectional view thereof.

As illustrated in FIG. 14, the measurement device 100g according to the seventh modification example is different from the measurement device 100 illustrated in FIG. 3 in that the measured object 200 has a substantially cylindrical shape and the light source 110, mirrors 146g, and the detection unit 160 are placed along an outer circumference of the measured object 200. Herein, the measured object 200 is, for example, a finger, a wrist, or an arm of the measured subject 210. Note that, in FIG. 14, the collimator 112, the polarizer 120, the retarder 130, the analyzer 150, and the support members 102 and 104 are not illustrated.

As illustrated in FIG. 14, in the measurement device 100g, the light source 110 and the detection unit 160 are placed to be substantially adjacent to each other, and the mirrors 146g are in contact with the measured object 200 at at least one point and are placed at positions corresponding to sides of a polygon. Herein, light emitted from the light source 110 is reflected by the plurality of mirrors 146g and passes through the outer circumference of the measured object 200, thereby reaching the detection unit 160.

Note that, in the measurement device 100g, the mirrors 146g do not need to be placed at the same height. For example, the mirrors 146g may be helically placed on the outer circumference of the measured object 200 by sequentially changing the height thereof.

With this configuration, the measurement device 100g can measure a finger, a wrist, an arm, and the like as the measured objects 200. In the case where a finger, a wrist, an arm, and the like are measured as the measured objects 200, in order to measure a state of a body fluid with high accuracy, it is preferable to transmit emitted light through a uniform biological tissue by avoiding a bone and a muscle whose compositions are different from a composition of another tissue. Therefore, it is preferable to place the mirrors 146g on the outer circumference of the measured object 200 as illustrated in FIG. 14 and to transmit emitted light through the outer circumference of the measured object 200 (for example, about 5 mm under the skin).

### 3. CONCLUSION

As described above, the measurement device 100 according to the embodiment of the present disclosure can remarkably increase an optical path length in the measured object 200 by causing light incident on the measured object 200 to be reflected by the mirrors 142 and 144. With this, even in the case where the measured object 200 is thin, the measurement device 100 can increase the optical path length by reflection and can therefore measure a state of a body fluid with high accuracy. Therefore, the measurement device 100 can be reduced in size and can be easily attached to a terminal part of a body of the measured subject 210, such as an earlobe, a finger, a wrist, or an arm.

The measurement device 100 according to the embodiment of the present disclosure may include the plurality of mirrors 142c and 144c provided in accordance with reflection positions of light in the measured object 200. With this configuration, reflection of light that is not on an optical axis of light emitted into the measured object 200 is suppressed, and therefore it is possible to prevent scattered light that has been multiply scattered and has moved around from entering the detector 160 and to improve measurement accuracy.

The measurement device 100 according to the embodiment of the present disclosure may include the plurality of mirrors 142c and 144c provided in accordance with the reflection positions of the light in the measured object 200, the light source 110, and the detector 160 so that the arrangement position and the direction of at least one thereof are changeable. The arrangement positions and the directions of the plurality of mirrors 142c and 144c, the light source 110, and the detector 160 which are provided so that the arrangement positions and the directions thereof are changeable may be independently controlled by the control unit 170. With this configuration, the measurement device 100 can cause the control unit 170 to optimize an optical path of light emitted into the measured object 200 so that the amount of received light of the detector 160 is maximized.

Note that the measurement device 100 according to the embodiment of the present disclosure can be applied to analysis of concentrations of components of various body fluids or blood and can also analyze information on a blood flow such as pulsation and pulse of blood.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

For example, although an optical path length of light emitted into the measured object 200 is increased by reflection caused by at least one mirror in the above embodiment, this technique can increase the optical path length of the light with another method. For example, the optical path length of the light emitted into the measured object 200 can be increased also by increasing an incident angle of the light emitted into the measured object 200 on an incident surface of the measured object 200.

In addition, the effects described in the present specification are merely illustrative and demonstrative, and not limitative. In other words, the technology according to the present disclosure can exhibit other effects that are evident to those skilled in the art along with or instead of the effects based on the present specification.

Additionally, the present technology may also be configured as below.

(1)

A measurement device, including:

a light source configured to emit light having a predetermined wavelength;

a polarizer configured to convert the light emitted from the light source to linearly polarized light;

a modulator configured to modulate a polarization direction of the linearly polarized light;

at least one mirror configured to reflect the light modulated in the modulator in a measured object;

an analyzer configured to separate, on the basis of a polarization direction of transmission light transmitted through the measured object, scattered light scattered in the measured object from the transmission light; and

a detector configured to detect the transmission light separated from the scattered light in the analyzer.

(2)

The measurement device according to (1),

wherein the mirror includes at least a pair of mirrors facing each other so that the measured object is sandwiched between the pair of mirrors.

(3)

The measurement device according to (2),

wherein the mirror includes a plurality of sets of mirrors, and

the mirrors are provided in accordance with respective reflection positions of the light in the measured object.

(4)

The measurement device according to (3),

wherein each of the mirrors is an independent movable mirror whose at least one of an arrangement position and a direction is changeable.

(5)

The measurement device according to any one of (1) to (4), further including

at least one of a light source rocking mechanism configured to rock the light source and a detector rocking mechanism configured to rock the detector,

wherein an optical path of the light transmitted through the measured object is controlled by the light source rocking mechanism and the detector rocking mechanism.

(6)

The measurement device according to (2),

wherein the pair of mirrors are provided so that an intersurface distance between a mirror surface provided on one side of the measured object and a mirror surface provided on the other side of the measured object is changeable.

(7)

The measurement device according to (2),

wherein each of the pair of mirrors is a half mirror.

(8)

The measurement device according to any one of (1) to (7),

wherein a pinhole is provided at a preceding stage of at least one of the analyzer and the detector.

(9)

The measurement device according to any one of (1) to (8), further including

a covering member configured to cover a measurement region of the measured object.

(10)

The measurement device according to (1),

wherein the mirror includes a plurality of mirrors placed along an outer circumference of the measured object.

(11)

The measurement device according to (4), further including

a control unit configured to control at least one of the arrangement position and the direction of the at least one mirror on the basis of an intensity of detection light detected by the detector.

(12)

The measurement device according to (5), further including

a control unit configured to control at least one of an arrangement position and a direction of at least one of the light source and the detector on the basis of an intensity of detection light detected by the detector.

(13)

The measurement device according to any one of (1) to (12), further including

a control unit configured to control output of the light source on the basis of an intensity of detection light detected by the detector.

(14)

The measurement device according to (6), further including

an analysis unit configured to analyze a measurement result by using an intensity of detection light detected by the detector on the basis of an incident angle of the light emitted by the light source on a horizontal surface and the intersurface distance between the pair of mirrors.

(15)

The measurement device according to (14), further including

a sensor configured to measure a state of the measured object or a state of an environment in which the measured object is placed,

wherein the analysis unit corrects the measurement result on the basis of the state measured by the sensor.

(16)

The measurement device according to (14) or (15),

wherein the measured object is a living body, and

the analysis unit analyzes at least one of a concentration of a component and pulsation of a body fluid of the living body by using the intensity of the detection light detected by the detector.

(17)

A measurement method, including:

converting light emitted from a light source configured to emit light having a predetermined wavelength to linearly polarized light;

modulating a polarization direction of the linearly polarized light;

reflecting the modulated light in a measured object;

separating, on the basis of a polarization direction of transmission light transmitted through the measured object, scattered light scattered in the measured object from the transmission light; and

detecting the transmission light separated from the scattered light.

### REFERENCE SIGNS LIST

100 measurement device
102, 104 support member
106 connection member
110 light source
112 collimator
120 polarizer
130 retarder
142, 144 mirror
150 analyzer
160 detector
170 control unit
190 analysis unit
200 measured object

1. A measurement device, comprising:
a light source configured to emit light having a predetermined wavelength;
a polarizer configured to convert the light emitted from the light source to linearly polarized light;
a modulator configured to modulate a polarization direction of the linearly polarized light;
at least one mirror configured to reflect the light modulated in the modulator in a measured object;
an analyzer configured to separate, on the basis of a polarization direction of transmission light transmitted through the measured object, scattered light scattered in the measured object from the transmission light; and
a detector configured to detect the transmission light separated from the scattered light in the analyzer.

2. The measurement device according to claim 1,
wherein the mirror includes at least a pair of mirrors facing each other so that the measured object is sandwiched between the pair of mirrors.

3. The measurement device according to claim 2,
wherein the mirror includes a plurality of sets of mirrors, and
the mirrors are provided in accordance with respective reflection positions of the light in the measured object.

4. The measurement device according to claim 3,
wherein each of the mirrors is an independent movable mirror whose at least one of an arrangement position and a direction is changeable.

5. The measurement device according to claim 1, further comprising
at least one of a light source rocking mechanism configured to rock the light source and a detector rocking mechanism configured to rock the detector,
wherein an optical path of the light transmitted through the measured object is controlled by the light source rocking mechanism and the detector rocking mechanism.

6. The measurement device according to claim 2,
wherein the pair of mirrors are provided so that an intersurface distance between a mirror surface provided on one side of the measured object and a mirror surface provided on the other side of the measured object is changeable.

7. The measurement device according to claim 2,
wherein each of the pair of mirrors is a half mirror.

8. The measurement device according to claim 1,
wherein a pinhole is provided at a preceding stage of at least one of the analyzer and the detector.

9. The measurement device according to claim 1, further comprising
a covering member configured to cover a measurement region of the measured object.

10. The measurement device according to claim 1,
wherein the mirror includes a plurality of mirrors placed along an outer circumference of the measured object.

11. The measurement device according to claim 4, further comprising
a control unit configured to control at least one of the arrangement position and the direction of the at least one mirror on the basis of an intensity of detection light detected by the detector.

12. The measurement device according to claim 5, further comprising
a control unit configured to control at least one of an arrangement position and a direction of at least one of the light source and the detector on the basis of an intensity of detection light detected by the detector.

13. The measurement device according to claim 1, further comprising
a control unit configured to control output of the light source on the basis of an intensity of detection light detected by the detector.

14. The measurement device according to claim 6, further comprising
an analysis unit configured to analyze a measurement result by using an intensity of detection light detected by the detector on the basis of an incident angle of the light emitted by the light source on a horizontal surface and the intersurface distance between the pair of mirrors.

15. The measurement device according to claim 14, further comprising
a sensor configured to measure a state of the measured object or a state of an environment in which the measured object is placed,
wherein the analysis unit corrects the measurement result on the basis of the state measured by the sensor.

16. The measurement device according to claim 14,
wherein the measured object is a living body, and
the analysis unit analyzes at least one of a concentration of a component and pulsation of a body fluid of the living body by using the intensity of the detection light detected by the detector.

17. A measurement method, comprising:
converting light emitted from a light source configured to emit light having a predetermined wavelength to linearly polarized light;
modulating a polarization direction of the linearly polarized light;
reflecting the modulated light in a measured object;
separating, on the basis of a polarization direction of transmission light transmitted through the measured object, scattered light scattered in the measured object from the transmission light; and
detecting the transmission light separated from the scattered light.
